(19) United States
(12) Patent Application Publication
(10) Pub. No.: US 2007/0197924 A1
(43) Pub. Date: Aug. 23, 2007

(54) Method and apparatus for determination of cardiac output from the arterial pressure pulse waveform

(76) Inventor: Michael Francis O'Rourke, New South Wales (AU)

Correspondence Address: Abelman Frayne and Schwab, 666 Third Avenue, New York, NY, 10017-5621, US

(21) Appl. No.: 10/591,742

(22) PCT Filed: Mar. 4, 2005
(86) PCT No.: PCT/AU05/00311

§ 371 Date: Aug. 30, 2006

(30) Foreign Application Priority Data
Mar. 5, 2004 (AU) 2004 901 161

(51) Int. Cl. A61B 5/02

(52) U.S. Cl. 600/504; 600/485

(57) ABSTRACT

A method for calculating aortic flow velocity from the directly or indirectly measured arterial, aortic, or carotid pressure and/or diameter waveform in which the reflected component of the pressure wave is excluded and the peak systolic flow velocity V is calculated from the amplitude P1 of the central pressure waveform using the formula (I) where C is the aortic pulse wave velocity.

### FIELD OF THE INVENTION

This invention relates to the determination of cardiac output (flow velocity or flow volume) from the heart through analysis of the pressure or diameter waveform in the upper limb (radial, brachial or subclavian artery) or in the neck (carotid artery).

### BACKGROUND ART

A clinical and scientific goal for many years has been to measure the cardiac output (blood flow ejected by the heart) from arterial pressure. The arterial pressure and diameter pulse waveform is created by this ejection, but on account of differences in arterial properties with age, differences in the pattern of flow ejection of the heart with age and with weakening of left ventricular muscle, and with change in hear rate under resting conditions, this goal has not been attained with precision.

Additional problems include the effects of wave travel and reflection in the arterial tree which cause variable amplification of the pressure pulse between central and peripheral arteries. This has been addressed by Kelly and Fitchett, J Am Coll Cardiol 1992;20:952-63, van Bortel et al., J Hypertens 2001 ;19:1037-44, Pauca, Kon and O'Rourke, Br J Anaesth 2004; 92: 651-7, and by other methods eg. U.S. Pat. No. 5,265,011.

An object of the present invention is to overcome the major problems created by aging and cardiac disease, as well as pressure pulse amplification in the more peripheral arteries, so that the radial or brachial arterial pulse, transformed to the aortic pulse, or the carotid or subclavian pulse in the neck, can be used to calculate the velocity of blood ejection from the heart into the aorta during each ejection (mean ejection velocity) and over a full beat (mean aortic flow velocity). Formulae utilising the known relationship of aortic diameter to body height and weight, or direct measurements of aortic dimensions by echocardiography can then be used to calculate blood volume from blood velocity.

### SUMMARY OF THE INVENTION

According to one aspect of the invention there is provided a method for calculating aortic flow velocity from the directly or indirectly measured arterial, aortic, or carotid pressure and/or diameter waveform in which the reflected component of the pressure wave is excluded and the peak systolic flow velocity V is calculated from the amplitude P1 of the central pressure waveform using the Waterhammer formula:

$$V = \frac{P 1}{1.05 * C}$$

where C is the aortic pulse wave velocity.

Preferably, ascending aortic pulse wave velocity as used in the waterhammer formula is measured directly, estimated from the delay from wavefoot to first systolic peak or shoulder, or taken from published data, from the US National Institute of Aging or another appropriate source for the subject's age.

Allowance is made for difference in aortic pulse wave velocity (PWV) with arterial pressure so as to apply normalised aortic PWV data to the individual.

Allowance may be made for the reduced aortic velocity in late systole with aging, caused by reduced ventricular contractility in late systole, and attributable to increased left ventricular (LV) load and LV hypertrophy or disease.

Allowance may be made for the further reduction in aortic velocity in late systole, caused by left ventricular weakening and relative change in the heart's pumping action from a “flow source” to a “pressure source”.

Average velocity in the aorta is preferably calculated for the period of ejection and the period of the cardiac cycle, and for other periods of time (eg. per second or per minute).

The preferred method of the invention permits the aortic flow velocity, normalised for that individual, to be expressed in terms of volume by multiplying by aortic cross-sectional area determined from echocardiography, other methods, of from tables, then to be expressed as volumetric cardiac output per minute.

According to another aspect of the invention there is provided a method for determining cardiac output comprising:

(i) determining the pressure waveform in the ascending aorta,

(ii) determining the amplitude (P1) of the initial peak of the aortic pressure waveform,

(iii) determining the aortic pulse wave velocity (C),

(iv) calculating the peak flow velocity (V) using the formula:

$$V = \frac{P 1}{1.05 * C}$$

(v) determining the mean systolic flow velocity (Vms) as a predetermined percentage of peak flow velocity (V) to allow for predetermined factors,

(vi) calculating the mean cycle flow velocity Vmc using the formula:

$$Vmc = Vms * \frac{period of systole}{period of cardiac cycle}$$

(vii) calculating the cardiac output by multiplying mean cycle velocity by the aortic cross-sectional area.

The pulse wave velocity may be normalised to means arterial pressure.

The mean systolic flow velocity may be reduced to allow for the effective heart rate. In a preferred form of the invention the mean systolic flow velocity is reduced by 0.9% for each beat per minute above 65 beats per minute.

### BRIEF DESCRIPTION OF THE DRAWINGS

FIG. 1 shows the aortic pressure waveform,

FIG. 2 shows the aortic flow waveform over one cardiac cycle,

FIG. 3a to 3f show the waveforms of the various steps of the method of calculating cardiac output according to one embodiment of the

### MODES FOR CARRYING OUT THE INVENTION

In a preferred form of the invention, the pressure waveform in the ascending aorta is determined by recording the carotid pressure or diameter waveform, calibrating this according to the methods of Kelly and Fitchett (J Am Coil Cardiol 1992;20:952-63) or van Bortel et al (J Hypertens 2001;19:1037-44) or similar methods, and taking this as a surrogate of the aortic pressure waveform.

Alternatively, a generalised transfer function may be applied to the calibrated pressure wave recorded invasively or non-invasively in the brachial or radial artery, using the process described in U.S. Pat. No. 5,265,011 or other appropriate methods.

Through use of differentials as described in the above publications or by other methods, the initial peak or shoulder of the aortic pressure waveform is identified and the height of this peak or shoulder (which typically is 90-120 msec after the pressure wavefoot) above the wavefoot itself is calculated. This is taken to represent the pressure wave generated by ventricular ejection before the return of significant wave reflection.

Alternatively, amplitude of this initial pressure peak or shoulder can be calculated directly from the radial or brachial pressure waves by exploiting the known relationship between brachiall radial and aortic augmentation (Nichols and O'Rourke, McDonald's Blood Flow in Arteries, 4^{th} ed., Arnold, London 1998; p.368 FIG. 16.20) and subtracting aortic augmentation from aortic pulse pressure.

The waterhammer formula (V=P/p.C) is used to calculate the velocity (V) of blood at peak ejection from the pressure wave unaffected by wave reflection (P) assuming density of blood (ρ)=1.05. The term C is aortic pulse wave velocity and the aortic pulse wave velocity multiplied by blood density is aortic characteristic impedance. This can be recorded directly as from delay in wavefeet between the carotid and femoral arteries, or indirectly from data prepared by Lakatta et al from the US National Institute of Aging (Lakatta EG. Cardiovascular Aging in Health. Heart Failure in the Elderly. In: Clinics in Geriatric Medicine 2000; 16:419-43), which link aorta pulse wave velocity with age, and show no gender difference.

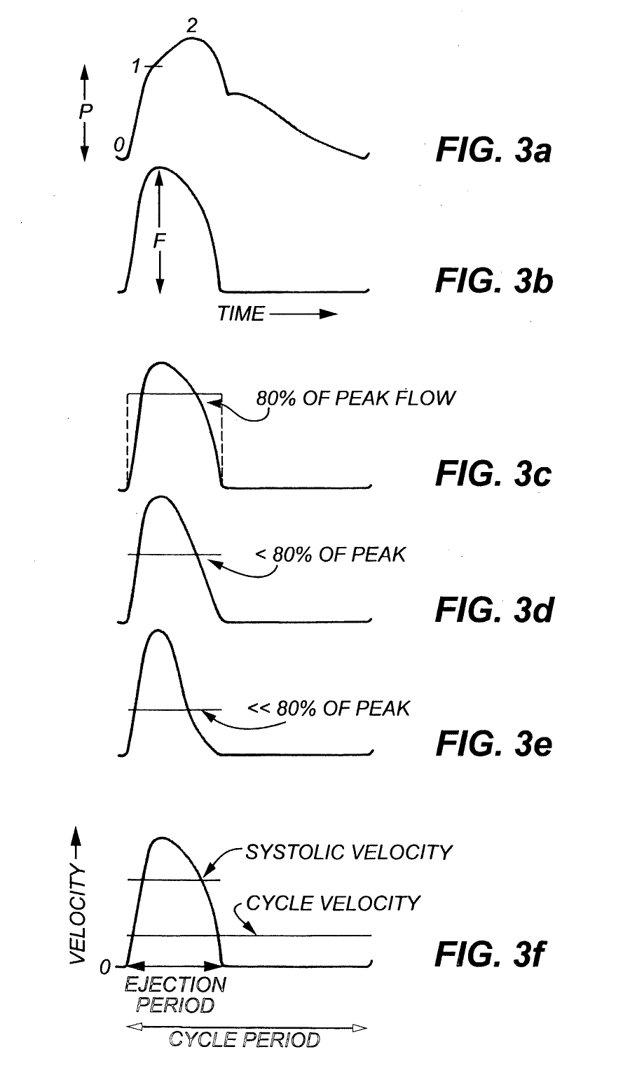

Other normal values e.g. Avolio et al., Circulation 1983;68:50-58; or Nichols and O'Rourke 1998, may be used instead. Examples of these data are shown in FIG. 3. [For the Lakatta data, the following formula is used:—C=8.52* age+222, where C is aortic pulse wave velocity in cm/sec]. Pulse wave velocity is normalised to mean arterial pressure of 100 mmHg by formula:—C adj=C−7.1*(100−mean pressure), derived from the known passive decrease 25 in aortic pulse wave velocity with fall in mean pressure described by Asmar et al, Hypertension 2001 ;38:921-6.

The duration of ejection from wavefoot to cardiac incisura is measured and compared to the total cycle length. This allows for differences in the duration of left ventricular contraction and relaxation as seen under different conditions eg. change in heart rate. The incisura is determined from the recorded radial, brachial, subclavian or carotid waveform, using differentials or other methods. The ejection duration is typically in the region of 250-350 msec, and relative duration of systole 30-40% of the cardiac cycle.

Assuming no flow in diastole, one then has to consider the shape of the aortic flow wave, and how this is influenced by aging and weakening of the ventricular muscle. In the first aspect, it is assumed that peak flow is achieved at the already identified shoulder and that flow ceases at the identified incisura. It is also assumed that the area under this curve in a healthy normal person under age 60 equates to a rectangle characterised horizontally by the duration of ejection and vertically by 80% of peak forward flow velocity. It is further assumed no flow in diastole. This step is necessary on account of intermittency of cardiac contraction and relaxation.

The effects of aging are allowed for by assuming relatively lower forward flow velocity in the latter part of systole after the early flow peak, as shown by Nichols et al (Am J Cardiol 1985;55:1179-84). As a first approximation, the value of 80% is taken to reduce by an absolute value of 10% for each decade over age 60 —ie. to 60% at age 80 and 50% at age 90. More precise approximations can be made when more normal human aging data becomes available.

The effect of impaired left ventricular contraction on the pulse waveform (Westerhof and O'Rourke. J Hypertension 1995;13:943-52) is allowed for by reducing the value for age calculated above by absolute 10% if LV ejection fraction (LVEF) is known to be between 25-40% (ie. from 80% at age 60 to 70%) and by absolute 20% if LV ejection fraction is known to be below 25% (ie. from 80% at age 60 to 60%). If cardiac failure is present and ejection fraction is not known, the first figure of 10% (as for LVEF 25-40%) is used.

Allowance can be made for the effective heart rate on flow waveform by reducing the predicted value of mean systolic velocity on mean cycle velocity by 0.9% for each beat per minute above 65 beats per minute. This correction is applied to the value already corrected for age and the presence of cardiac failure.

Calculated flow velocity is thus determined per ejection (i.e. per beat). This is normalised for body size since the waterhammer formula relates pressure change to velocity change. In each individual, velocity can be converted to volume flow (stroke volume in mis/beat) from measurement of aortic cross-sectional diameter and area by ultrasound, or from the nomogram from Lakatta et al (Lakatta EG. Cardiovascular Aging in Health. Heart Failure in the Elderly. In: Clinics in Geriatric Medicine 2000;16:419-43) which relates body height and weight to aortic cross-sectional area.

From the Lakatta data, the formula:
D=(0.0654*age)+12.63
can be used where D is diameter per square meter body surface area.

Body surface area (BSA) may be calculated from the equation of Du Bois and Du Bois:
BSA=Mass^{0.426}(Kg)*Height^{0.725}(cm)*71.84.

See Dubois and Dubois (Arch.) Intern. Med 1916; 17:863) or as converted into nomograms in the Geigy Scientific Tables (—see FIG. 5).

Cardiac output may be calculated as mean aortic flow velocity times calculated aortic cross-sectional area. On account of difficulties in accurately measuring or estimating aortic cross-sectional area, a preference is given for expression of average flow as velocity over the whole cycle (this is the same as average velocity flow over a full minute).

The method described has other aspects, and it will be appreciated that variations and additions are possible within the spirit and scope of the invention. Previous applications of this type of method have used the waterhammer formula on central or peripheral pressure pulse waveforms. The specific novelty of this approach is that it takes account of the change in left ventricular ejection pattern which occurs with aging (as a consequence of early wave reflection and late systolic pressure augmentation), and the progressive change of the heart from a “flow source” to a “pressure source” with aging as afterload increases (Nichols et al. Am J Cardiol 1985;55:1179-84), and other further change which occurs as the ventricular muscle weakens through ventricular hypertrophy or from intrinsic cardiac (eg. coronary) disease (O'Rourke M F. Blood Pressure 1994;3:33-37). The approach also concentrates on scaled values of peak and mean flow in terms of linear velocity rather than volume flow.

Other aspects of the invention allow direct measurement of aortic pulse wave velocity, or its estimation from the time to return of the reflected wave (London et al. Hypertension 1992;20:10-19), instead of from the NIA or other tables. Other aspects entail use of the calibrated carotid or upper limb pressure pulse waveform to determine the impulse generated by ejection (without reflection) and of ultrasonic or other techniques to calculate aortic cross-sectional area. On account of different systematic effect of age on the proximal aorta that on the truncal arteries, it may be necessary to introduce a scaling term in the equations used to calculate peak velocity from pressure under different conditions. It may also be necessary to vary the constants presently proposed to describe the effect of aging and of heart failure on the arterial pulse.

It is desirable to allow for change in the flow waveform contour at different heart rates at rest by reducing the calculated average flow velocity below that initially calculated when heart rate increases above 65 beats/minute.

Measurement of cardiac output or cardiac index (scaled to body dimensions as achieved here) is an important clinical measure, being increased in exercise and pregnancy, with thyroid overactivity, and with some forms of “hypertension”, including “white coat hypertension”. Cardiac index is reduced with blood or fluid loss, with pulmonary embolism and cardiac failure from multiple causes. Since the major function of the heart is to pump blood, measurement of the amount pumped is a valuable clinical sign. To determine this simply with logical physiological processes is an important advance.

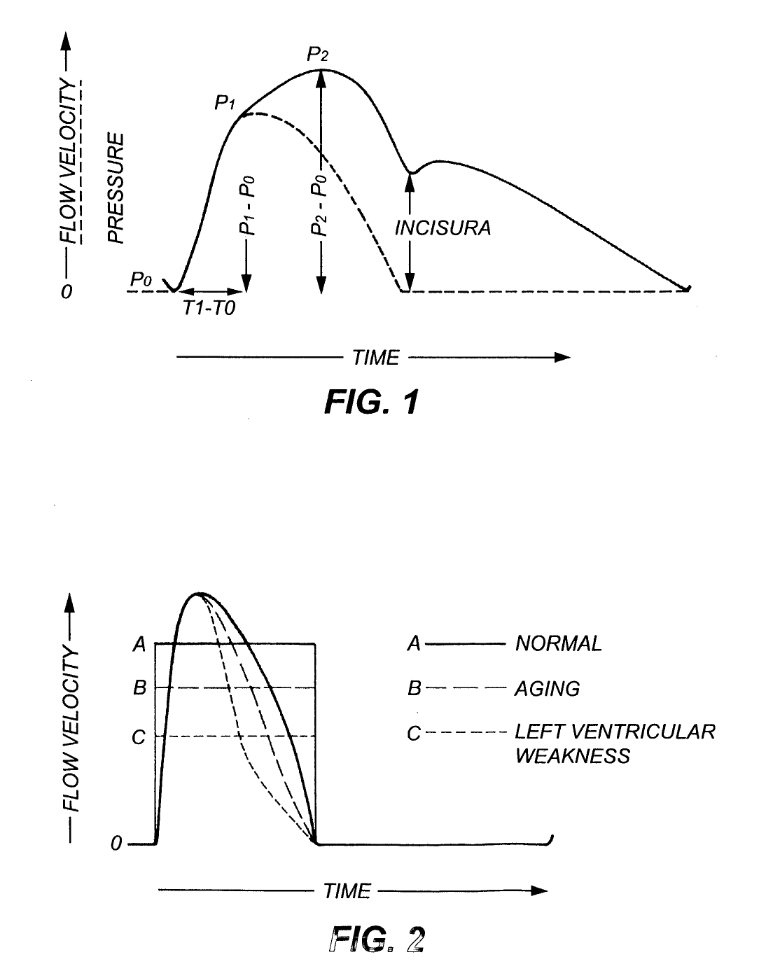

FIG. 1 shows a central (aortic or carotid) pressure waveform recorded directly or synthesised from a peripheral pressure waveform. The point 0 is the foot of the wave from which pressure rises smoothly up to a localised peak or shoulder at point 1, some 90-120 msec after the foot of the wave. Pressure may rise further after this point 1, but then declines to an inflection or incisura typically some 250-350 msec after the wavefoot. This incisura denotes closure of the aortic valve and the end of ventricular ejection.

The pressure rise from point 0 to point 1 (P1 -P0) is determined by the flow ejection in the time T1-T0, by aortic pulse wave velocity and blood density according to the waterhammer formula. The flow velocity change from zero at the wavefoot to its peak and back to zero at the incisura is represented by the broken line. The peaks of pressure and flow correspond at point 1.

The aortic flow velocity waveform shown in FIG. 2 in systole normally approximates to a rectangle (dotted lines) of base corresponding to ejection duration and height corresponding to 80% of peak flow. This alters with aging as left ventricular load increases and the ventricle hypertrophies. Ejection duration may increase, but the patterns of late systolic flow changes such that there is low velocity in later systole and average systolic flow typically falls to 60% of peak flow.

In cardiac failure the same phenomenon is seen with lower flow in late systole. The period of systole may also be decreased but this is directly measurable as reduction in ejection duration, and of ejection duration/duration of cardiac cycle. Presently, the subtle change in the flow pattern can only be inferred from the lesser degree of augmentation than expected in late systole.

A nomogram of the relationship between aortic pulse wave velocity and age is set forth in NIA studies (Lakatta EG. Cardiovascular Aging in Health. Heart Failure in the Elderly. In: Clinics in Geriatric Medicine 2000;16:419-43).

A nomogram of the relationship between aortic diameter and body surface area is set forth in NIA studies (Lakatta EG. Cardiovascular Aging in Health. Heart Failure in the Elderly. In: Clinics in Geriatric Medicine 2000; 16:419-43).

A nomogram for the calculation of body surface area is included in the Geigy Scientific Tables.

The steps of one method of the invention are as follows with reference to FIGS. 3a to 3f:

Step 1: calculation of pressure rise in aorta caused by peak flow ejection (P1) (multiple methods possible),—FIG. 3a.

Step 2: calculation of peak flow velocity (F) corresponding to (P1) using water hammer formula. Determination of aortic pulse wave velocity from normative data, and correcting this to a value appropriate to the individual's mean arterial pressure,—FIG. 3b.

Step 3: calculation of mean flow velocity in systole, by assuming that mean systolic flow=80% of peak flow—FIG. 3c.

Step 4: allowance for effect of age on flow pattern, with reduced flow in late systole causing mean systolic flow to be <80% of peak, FIG. 3d.

Step 5: allowance for effect of heart failure, with flow in late systole further reduced at any given age to <<80% of peak,-FIG. 3e.

Step 6: allowance for heart rate by further reduction in systolic flow to <<<80% with heart rate over 65/minute.

Step 7: calculation of mean cycle flow velocity as (mean systolic velocity)×Z, —FIG. 3f.

$$where Z = \frac{period of systole}{period of cycle}$$

Step 8: calculation of volume flow from mean cycle velocity and aortic cross sectional area as cardiac output.

### EXAMPLE

Subject: Male, 67 years, height 168 cm, weight 76Kg.
Mean blood pressure: 87 mm Hg
Initial peak amplitude (P1): 33 mm Hg
Heart rate: 67 bpm
Period of systole/period 36% of cardiac cycle:

Utilizing the steps set forth above, the cardiac output in litres/minute is calculated from the initial peak amplitude (P1) as follows:

$$\begin{matrix}( i ) Pulse wave velocity ( C ) = 8.52 * Age + 222 \\ = 8.52 * 67 + 222 \\ = 792.7 cm \text{/} sec\end{matrix}$$

$$\begin{matrix}( ii ) \begin{matrix}\begin{matrix}Pulse wave velocity \\ normalised to mean\end{matrix} \\ blood pressure\end{matrix} = C - 7.1 ( 100 - mbp ) \\ = 792.7 - 7.1 ( 100 - 87 ) \\ = 700.4 cm \text{/} sec\end{matrix}$$

$$\begin{matrix}( iii ) Peak flow velocity ( V ) = \frac{P 1}{1.05 * C} \\ = \frac{33 ( 980 * 1.36 )}{1.05 * 700.4} \\ = 59.8 cm \text{/} sec\end{matrix}$$

$$\begin{matrix}( iv ) \begin{matrix}\begin{matrix}Age adjustment factor \\ ( normal cardiac function )\end{matrix} \\ ( AAF )\end{matrix} = 80 \% minus 1 \% for \\ each year over 60 \\ = 80 \% - 7 \% \\ = 73 \%\end{matrix}$$

$$\begin{matrix}( v ) Mean systolic flow velocity = 73 \% Peak flow velocity \\ = 0.73 * 59.8 \\ = 43.65 cm \text{/} sec\end{matrix}$$

$$\begin{matrix}( vi ) Heart rate adjustment = ( Heart rate - 65 ) 90 \% \\ = ( 67 - 65 ) 0.9 \\ = 1.8 \%\end{matrix}$$

$$\begin{matrix}( vii ) \begin{matrix}Adjusted mean systolic \\ flow velocity ( Vms )\end{matrix} = ( 100 \% - 1.8 \% ) * 43.65 \\ = 42.86 cm \text{/} sec\end{matrix}$$

$$\begin{matrix}( viii ) \begin{matrix}Mean cycle flow \\ velocity ( Vmc )\end{matrix} = Vms * \frac{period of systole}{period of cardiac cycle} \\ = 42.86 * 36 \% \\ = 15.43 cm \text{/} sec\end{matrix}$$

$$( ix ) Aortic cross \text{-} sectional area ( A c / s ) = 7.8$$

$$\begin{matrix}( x ) Cardiac output = Vmc * A c / s \\ = 15.43 * 7.8 * 60 \\ = 120.3 ml \text{/} sec * 60 \\ = 7.22 Litres \text{/} minute\end{matrix}$$

Various modifications may be made in details of the method without departing from the scope and ambit of the invention.

1. A method for calculating aortic flow velocity from the directly or indirectly measured arterial, aortic, or carotid pressure and/or diameter waveform in which the reflected component of the pressure wave is excluded and the peak systolic flow velocity V is calculated from the amplitude P1 of the central pressure waveform using the formula
$$V = \frac{P 1}{1.05 * C}$$
where C is the aortic pulse wave velocity.

2. A method according to claim 1 wherein the aortic pulse wave velocity is measured directly, estimated from the delay from wavefoot to first systolic peak or shoulder, or taken from published data, then normalised to mean pressure.

3. A method according to claim 1 wherein allowance is made for the reduced aortic velocity in late systole with aging, caused by reduced ventricular contractility in late systole, and attributable to increased left ventricular (LV) load and LV hypertrophy or disease.

4. A method according to claim 3, wherein allowance is made for the further reduction in aortic velocity in late systole, caused by left ventricular weakening and relative change in the heart's pumping action from a flow source to a pressure source.

5. A method according to claim 1 wherein the average velocity in the aorta is calculated for the period of ejection and the period of the cardiac cycle.

6. A method according to claim 1 in which the aortic flow velocity, normalised for that individual, is expressed in terms of volume by multiplying by aortic cross-sectional area and expressed as volumetric cardiac output per minute.

7. A method for determining cardiac output comprising:
(i) determining the pressure waveform in the ascending aorta,
(ii) determining the amplitude (P1) of the initial peak of the aortic pressure waveform,
(iii) determining the aortic pulse wave velocity (C),
(iv) calculating the peak flow velocity (V) using the formula:
$$V = \frac{P 1}{1.05 * C}$$
(v) determining the mean systolic flow velocity (Vms) as a predetermined percentage of peak flow velocity (V) to allow for predetermined factors,
(vi) calculating the mean cycle flow velocity Vmc using the formula:
$$Vmc = Vms \times \frac{period of systole}{period of cardiac cycle}$$
(vii) calculating the cardiac output by multiplying mean cycle velocity by the aortic cross-sectional area.

8. A method according to claim 7 wherein the pressure waveform in the ascending aorta is determined by recording the carotid pressure or diameter waveform and using this waveform to provide an estimate of the ascending aorta pressure waveform.

9. A method according to claim 7 wherein the pressure waveform in the ascending aorta is determined by applying a generalised transfer function to a calibrated pressure wave recorded invasively on non-invasively in the brachial or radial artery.

10. A method according to claim 7 wherein the amplitude P1 of the aortic pressure waveform is determined by identifying the initial peak or shoulder of the aortic pressure waveform and calculating the height of this peak about the foot of the waveform.

11. A method according to claim 7 wherein the amplitude P1 of the aortic pressure waveform is calculated directly from the radial or brachial pressure waves using the relationship between brachial/radial and aortic augmentation and subtracting aortic augmentation from aortic pulse pressure.

12. A method according to claim 7 wherein the aortic pulse wave velocity (C) is determined by recording the delay in wavefeet between the carotid and femoral arteries.

13. A method according to claim 7 wherein the aortic pulse wave velocity (C) is determined from the age of the patient irrespective of gender.

14. A method according to claim 7 wherein the aortic pulse wave velocity (C) is calculated using the formula:
C=8.52*Age+222.

15. A method according to claim 7 wherein the mean systolic flow velocity (Vms) is less than 80% of the peak flow velocity (M).

16. A method according to claim 7 wherein the predetermined factors are age, impaired left ventricular contraction and /or heart failure.

17. A method according to claim 16 wherein the allowance for the age factor assumes relatively lower forward flow velocity in the late part of systole after early peak flow.

18. A method according to claim 7 wherein the mean systolic flow velocity (Vms) is determined by assuming that mean systolic flow equals 80% of peak flow to take account of intermittency of cardiac contraction and relaxation.

19. A method according to claim 18 wherein the mean systolic flow velocity (Vms) is reduced to allow for the effects of aging by assuming relatively lower forward flow velocity in the later part of systole after the early flow peak.

20. A method according to claim 19 wherein the age effect reduction is an absolute value of 10% for each decade over age 60.

21. A method according to claim 19 wherein the mean systolic flow velocity is further reduced to allow for the effects of impaired left ventricular contraction on the aortic pressure waveform by absolute 10% if the left ventricular ejection fraction is known to be between 25 to 40% and by an absolute 20% if the left ventricular ejection fraction is known to be below 25%.

22. A method according to claim 21 wherein the mean systolic flow velocity is reduced by 10% if cardiac failure is present and the left ventricular fraction is not known.

23. A method according to claim 7 wherein the period of systole is determined by measuring the duration of ejection from the foot of the aortic waveform to cardiac incisura.

24. A method according to claim 7 wherein the aortic cross-sectional area is measured by ultra-sound.

25. A method according to claim 7 wherein the aortic cross-sectional area is determined from the correlation of body height and weight to aortic cross-sectional area.

26. A method according to claim 25 wherein the aortic cross-sectional area is calculated from the diameter per square meter body surface area (D) using the formula:
D=0.0654×Age+12.63
and establishing the square meter body surface area from the body height and weight.

27. A method according to claim 7 wherein the mean systolic flow velocity is reduced to allow for the effective heart rate.

28. A method according to claim 27 wherein the mean systolic flow velocity is further reduced by 0.9% for each beat per minute above 65 beats per minute.

29. A method according to claim 7 wherein the pulse wave velocity is normalised to mean arterial pressure.

30. A method according to claim 29 wherein the normalised pulse wave velocity (C adj) is calculated using the formula:
C adj=C−7.1(100−mbp)
where mbp is the mean arterial pressure.
